(19) United States
(12) Patent Application Publication
(10) Pub. No.: US 2017/0173257 A1
(43) Pub. Date: Jun. 22, 2017

(54) UTEROTUBAL IRRIGATION TECHNIQUE AND DEVICE

(71) Applicant: NVISION MEDICAL CORPORATION, Saratoga, CA (US)

(72) Inventors: Surbhi SARNA, Saratoga, CA (US); Albert CHIN, Saratoga, CA (US)

(21) Appl. No.: 15/127,701

(22) PCT Filed: Mar. 20, 2015
(86) PCT No.: PCT/US2015/021858

§ 371 Date: Sep. 20, 2016

Related U.S. Application Data
(60) Provisional application No. 61/968,226, filed on Mar. 20, 2014.

(51) Int. Cl. A61M 3/02; A61B 10/02; A61B 17/42

(52) U.S. Cl. CPC A61M 3/0295; A61M 3/0283; A61B 17/42; A61M 3/0254; A61M 3/0287; A61B 10/0291; A61B 2017/4216; A61B 2017/4233; A61M 2210/1425

(57) ABSTRACT

An uterotubal irrigation system and process for implanting hysterosalpingogram (HSG) procedures is provided. The uterotubal irrigation system serially irrigates and evacuates fluid in the uterus and into the Fallopian tubes, for the collection of cells from the Fallopian tubes. The uterotubal irrigation system has a partially flexible cannula for introduction through the cervical os into the uterine cavity. The cannula has an enlarged external plug proximal to the distal tip of the cannula, to occlude the os and permit infusion of fluid into the uterus. The cannula has an internal tube for irrigation, and an external sheath with multiple slits near the distal end of the cannula. An uterotubal irrigation system is provided that includes a catheter with two opposing outlet openings on a distal tip of the catheter that injects an irrigation fluid in two opposing jets that splay out laterally toward the openings of a patient's Fallopian tubes.

### RELATED APPLICATIONS

This application claims priority benefit of U.S. Provisional Application Ser. No. 61/968,226 filed Mar. 20, 2014; the contents of which are hereby incorporated by reference.

### FIELD OF THE INVENTION

The present invention in general relates to medical devices and in particular to a device and method to serially irrigate fluid followed by evacuation of the fluid in the uterus, with extension of the fluid into the Fallopian tubes, for the purpose of collecting cells from the Fallopian tubes.

### BACKGROUND OF THE INVENTION

Ovarian cancer is a cancer that begins in an ovary, and is the result of the development of abnormal cells that have the ability to invade or spread to other parts of the body. In 2012, ovarian cancer occurred in 239,000 women and resulted in 152,000 deaths worldwide, which made ovarian cancer the seventh most common cancer and the eighth most common cause of death from cancer in women. Ovarian cancer is disproportionately deadly because this type of cancer lacks any clear early detection or screening test, meaning that most cases of ovarian cancer are not diagnosed until they have reached advanced stages. Thus, ovarian cancer screening is of high clinical interest because the disease is not typically detectable at its early stages, when it is the most curable.

Occasionally, ovarian tumor cells may migrate into the uterus. Thus, it would be useful to have a device that may irrigate a portion of the Fallopian tubes on both sides, and collect the irrigation fluid for cell analysis in the search for an ovarian malignancy. Furthermore, ovarian cancer cells may proceed in a retrograde direction from the ovary into the Fallopian tube. It is also thought that some ovarian cancers have their origins in the Fallopian tube. Therefore, the ability to flush fluid into the Fallopian tube and to collect this fluid is desirable from a diagnostic standpoint.

Introduction of fluid into the uterus is commonly performed for a hysterosalpingogram (HSG), a diagnostic radiologic procedure involving introduction of contrast material under pressure into the uterus, to cause the contrast to flow into the Fallopian tubes for visualization of the uterus and Fallopian tubes. However, retrieval of injected fluid is extremely difficult or impossible to perform. The uterus is a muscular organ with a tiny intraluminal volume (approximately 3-5 cc) with a collapsible structure, and the Fallopian tube has a small diameter (approximately 1 mm at its proximal portion). At the junction of the uterus and the Fallopian tube is the uterotubal junction, where the lumen is 0.3 to 0.5 mm in diameter. Thus, irrigation requires significant pressure to cause injected fluid to track from the uterus into the tube, and attempts to retrieve the injected fluid are generally unsuccessful. When a vacuum is drawn on an intrauterine catheter, the uterus collapses around the catheter and prevents withdrawal of injected fluid.

Infusion catheters are used for hysterosalpingography. During hysterosalpingography, infusion catheters are advanced into the uterus, while an enlarged portion of the catheter seals against the cervical os to allow fluid pressure to be developed in the uterus. The cervical sealing portion of the catheter may be a balloon, a solid dilated structure on the catheter body, or a foam stopper. Infusion catheters are designed to inject fluid, and fluid retrieval is not contemplated or performed with these catheters. Thus, there exists a need for a device and method to serially irrigate fluid, followed by evacuation of the fluid in the uterus, with extension of the fluid into the Fallopian tubes, for the purpose of collecting cells from the Fallopian tubes for examination and analysis, while also avoiding the pain and discomfort experienced by patients during the diagnostic procedure.

### SUMMARY OF THE INVENTION

An uterotubal irrigation system is provided that includes a cannula with an external sheath that has a larger inner diameter than an external diameter of an irrigation tube positioned within the sheath so as to form an evacuation channel between the external sheath and the irrigation tube along a length of the cannula, and where a distal end of the sheath is connected to a second distal end of the irrigation tube; a syringe in fluid communication via an irrigation port with the irrigation tube and a fluid reservoir, the said syringe having a primary vacuum port connected to a primary vacuum line connected to a vacuum source; an evacuation port connecting the cannula to the syringe; a second vacuum line that is smaller then the primary vacuum line in fluid communication with the evacuation channel and a collection tube, the collection tube for storing a fluid evacuated from a patient's uterus following injection of the fluid that has been previously stored in the fluid reservoir; and two or more slits formed in a distal end of the sheath, the two or more slits expanding outward with the retraction of the irrigation tube to form an evacuation basket to support uterine walls of the patient's uterus under an applied vacuum during fluid evacuation from the uterus. The syringe further includes a plunger having a plunger seal, where the plunger is biased with a spring so that the plunger seal is positioned to block the primary vacuum port into the syringe, and a vacuum produced by the vacuum source is pulled through the second vacuum line to evacuate the injected fluid via the evacuation basket and the evacuation channel.

A process of using the uterotubal irrigation system is provided that includes inserting a cannula into the patient's uterus; expanding the evacuation basket by retracing the irrigation tube; injecting a fluid into the patients uterus; evacuating the fluid from the patient's uterus and retrieving and collecting the fluid in the collection tube; and wherein the injecting and evacuating are controlled with the depression of the syringe plunger to modulate the degree of vacuum. During the process of using the uterotubal irrigation system, the injecting and evacuating are staggered to serially and repetitively inject and retrieve multiple fluid aliquots to provide a sufficient fluid volume and number of sample cells for evaluation. In a specific embodiment the process is a hysterosalpingogram (HSG) procedure.

An uterotubal irrigation system is provided that includes a catheter with two opposing outlet openings on a distal tip of the catheter that injects an irrigation fluid in two opposing jets that splay out laterally toward the openings of a patient's Fallopian tubes when the catheter is inserted in the uterus of the patient, where the two opposing outlets are angled toward the openings to the patient's Fallopian tubes, an occlusion balloon or a plug that is situated on a wall of the catheter at a distance between 1.5 to 2.5 centimeters proximal to the distal tip of the catheter that is inflated to seal the patient's cervical os prior to insertion of the catheter distal tip into the patient's uterus; and a collection inlet proximal to the distal catheter tip for collecting the injected irrigation fluid.

A process of using the uterotubal irrigation catheter system is provided that includes inflating the occlusion balloon; inserting the catheter into a patient's uterus; injecting a fluid into the uterus; and evacuating the fluid from the patient's uterus and retrieving and collecting the fluid at the collection port. The process of injecting and evacuating are staggered to serially and repetitively inject and retrieve multiple fluid aliquots to provide a sufficient fluid volume and number of sample cells for evaluation. In a specific embodiment the process is a hysterosalpingogram (HSG) procedure.

### BRIEF DESCRIPTION OF THE DRAWINGS

FIG. 1 is a picture of a uterotubal irrigation system according to an embodiment of the invention;

FIG. 2 is a close-up picture of an irrigation cannula of the uterotubal irrigation system of FIG. 1 according to an embodiment of the invention;

FIG. 3 is a close-up picture of an evacuation basket on the tip of the irrigation cannula of the uterotubal irrigation system of FIG. 1 according to an embodiment of the invention;

FIG. 4 is a schematic block diagram of the uterotubal irrigation system of FIG. 1 in an injection mode according to an embodiment of the invention;

FIG. 5 is a schematic block diagram of the uterotubal irrigation system of FIG. 1 in a collection mode according to an embodiment of the invention;

FIGS. 6A and 6B are schematic block diagrams of irrigation cannula of FIG. 1 showing the retraction of the irrigation tube to expand the distal evacuation basket according to an embodiment of the invention;

FIG. 7A is a schematic block diagram of a cell collection irrigation catheter according to an embodiment of the invention;

FIG. 7B is a cross-sectional view along line A-A of the cell collection irrigation catheter of FIG. 7A;

FIG. 8 is a schematic block diagram of the cell collection irrigation catheter of FIGS. 7A and 7B showing fluid flow paths in an irrigation and collection mode when inserted in a uterus according to an embodiment of the invention; and

FIG. 9 is schematic block diagram of a pressure limiting injection device for use with the cell collection irrigation catheter of FIG. 1A and FIG. 7A according to an embodiment of the invention.

### DESCRIPTION OF THE INVENTION

The present invention has utility as an uterotubal irrigation system and process for implanting hysterosalpingogram (HSG) procedures. Embodiments of the inventive uterotubal irrigation system serially irrigate fluid followed by evacuation of the fluid in the uterus, with extension of the fluid into the Fallopian tubes, for the purpose of collecting cells from the Fallopian tubes. Due to the tiny volume of the uterus, a single injection of several cc's of fluid followed by evacuation will yield a minimal amount of fluid for analysis. It is thus necessary to repetitively inject and retrieve multiple fluid aliquots to provide sufficient fluid volume and sample cells for evaluation. It is also important to stagger the steps of fluid injection and fluid retrieval, otherwise a concomitant injection and evacuation will prevent fluid from ever entering the Fallopian tube.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

Embodiments of the uterotubal irrigation system provide a partially flexible cannula that is introduced through the cervical os into the uterine cavity. The inventive cannula has an enlarged external plug of approximately 2 cm that is proximal to the distal tip of the cannula, to occlude the os and permit infusion of fluid into the uterus. The inventive cannula has an internal tube for irrigation, and an external sheath with multiple slits near the distal end of the cannula. The distal tip of the external sheath is attached to the distal end of the irrigation tube. In specific embodiments, the inner diameter of the external sheath is approximately 0.5 mm greater than the outer diameter of the irrigation tube. The irrigation tube passes through a sliding seal on the proximal end of the external sheath. An irrigation port on the proximal end of the irrigation tube permits fluid infusion, while an evacuation port on the proximal end of the external sheath allows evacuation of fluid via the space between the outer diameter of the irrigation tube and the inner diameter of the external sheath. When the irrigation tube is retracted relative to the external sheath, a series of slits on the sheath at the distal tip of the cannula expand outward to form a basket to maintain the uterine cavity and prevent the uterine cavity from collapse due to a vacuum draw during evacuation of fluid for cell analysis.

Embodiments of the inventive uterotubal irrigation system provide serial fluid injection followed by fluid evacuation. The injection and evacuation modes of embodiments of the inventive system are controlled with the depression of a syringe plunger to modulate the degree of vacuum exhibited in the evacuation mode of the irrigation cannula. The system utilizes a port in the side of the syringe body, where upon full retraction of the syringe plunger against a stop set at a predetermined volume (e.g., 5 cc), the plunger seal covers and seals the port. Parallel vacuum lines are present in the system, where one vacuum line is a small diameter (approximately 0.5 mm) line that connects to the evacuation port on the external sheath of the irrigation cannula, and the other vacuum line is a large diameter (approximately 5-10 mm along the majority of its length) line that extends to the vacuum source. A collection tube is positioned in-line between the evacuation port on the irrigation cannula and the large diameter pressure line. When the spring loaded syringe plunger is completely retracted, during refilling of the syringe from a fluid source, the plunger seal closes off the vacuum port into the syringe from the large vacuum line, and a vacuum is pulled through the small diameter line to evacuate fluid via the expanded basket on the irrigation cannula. When the syringe plunger is depressed to inject fluid, the port on the side of the syringe connected to the large diameter vacuum line is opened. During plunger depression, the majority of vacuum flow is derived from the large diameter line, with its low fluid resistance, and a minimal vacuum is experienced in the small diameter line to drain the fluid as the fluid is being injected by the syringe. The flow rate and negative pressure provided by the vacuum source is also maintained at a moderate level to render the system functional. A one-way valve is present at the fluid irrigation source, so that fluid may only be introduced into the syringe and out of the irrigation cannula upon syringe depression and retraction.

An inventive embodiment of the uterotubal irrigation system is provided as a catheter for cell sampling that has two outlet openings on the catheter distal tip that inject irrigation fluid in two opposing jet streams that splay out laterally toward the os or openings of both Fallopian tubes. The two separate opposing outlets on the distal tip of the catheter are angled toward the openings to the Fallopian tubes, where the irrigation channels within the catheter bend outward toward the outlet openings. In operation an occlusion balloon or a plug that is situated at a distance between 1.5 to 2.5 centimeters proximal to the tip of the catheter is inflated to seal the cervical os prior to insertion of the catheter tip into the uterus. In a specific embodiment, the occlusion balloon or a plug is situated at a distance of 2.0 centimeters proximal to the tip of the catheter. The occlusion balloon seals the cervical os during the irrigation and fluid collection process. The injected irrigation fluid proceeds a distance into both Fallopian tubes, and the fluid then circulates back into the uterine cavity, where the fluid exits via a collection port in the catheter. In an inventive embodiment, the collection inlet is approximately 1 cm proximal to the distal catheter tip. The retrieved irrigation fluid undergoes cytologic examination to detect the presence of malignant cells.

Embodiments of the catheter based uterotubal irrigation system provide serial fluid injection followed by fluid evacuation. An inventive fluid injection device is provided that may be used in conjunction with the inventive cell collection irrigation catheter to limit the amount of pressure used for injection. The use of embodiments of the inventive fluid injection device may be used to avoid the pain and discomfort experienced by the patient during the diagnostic procedure. Embodiments of the pressure limiting fluid injection device have a syringe plunger that is composed of a compression spring connected to the distal sealing plunger face. A threaded plunger advances to compress the compression spring. The threaded plunger contains a groove along its length that is keyed by a pin that extends through the syringe body into the groove. A drive disc is rotatably fixed on the proximal end of the syringe body, and the drive disc contains internal threads that mate with the threaded plunger. When the drive disc is rotated, the threaded plunger moves forward to compress the fluid inside the syringe. The maximal pressure that may be developed by the syringe is determined by a clutch disc that lies coaxially outside the drive disc. At a predetermined amount of torque, the clutch disc slips relative to the drive disc. The torque setting may be set by adjusting the friction that exists between the clutch disc and the drive disc. In a specific embodiment, one or more clutch adjustment screws extending through the clutch disc may be tightened down on the drive disc, so that the torque exerted on the threaded plunger will be limited to a given level. This in turn limits the degree of compression exerted by the spring, thus limiting the injection pressure. The pressure limiting injection device incorporates the compression spring for energy storage, such that continuous rotation of the clutch disc is unnecessary for fluid injection. Rather, the clutch disc is rotated to bring the syringe to the desired injection pressure level, and then rotated at intervals as necessary to re-pressurize the system. In a specific embodiment the full retraction of the syringe plunger provides a predetermined volume of irrigation fluid (e.g., 5 cc).

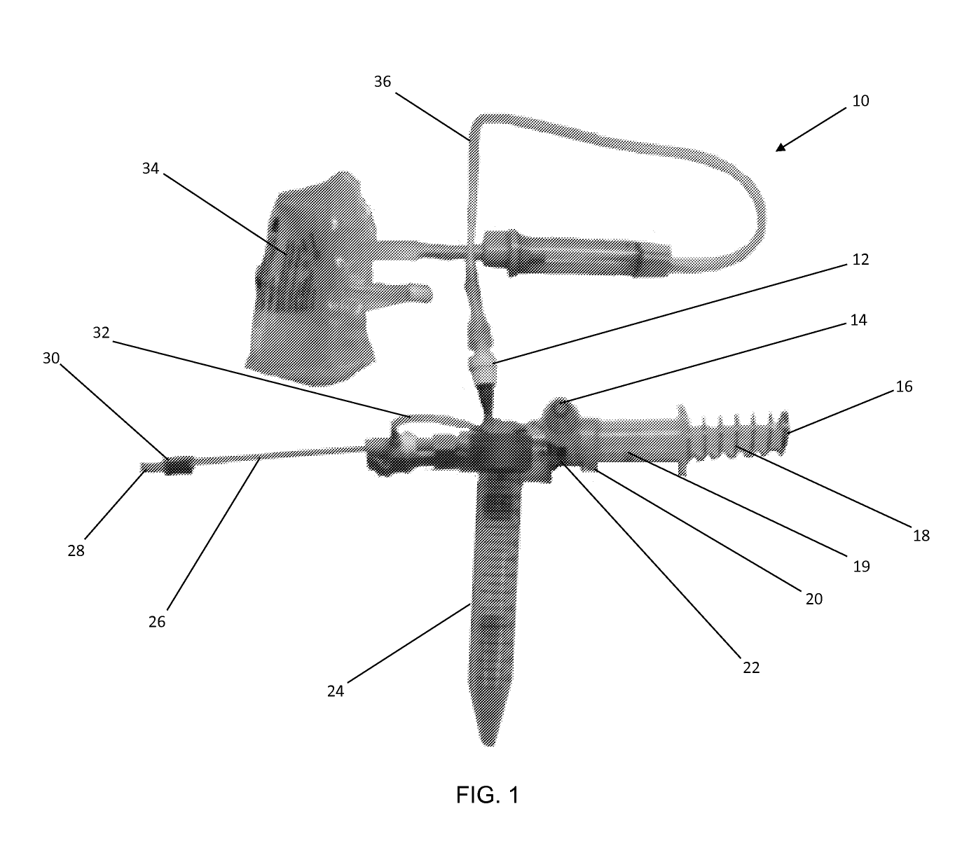

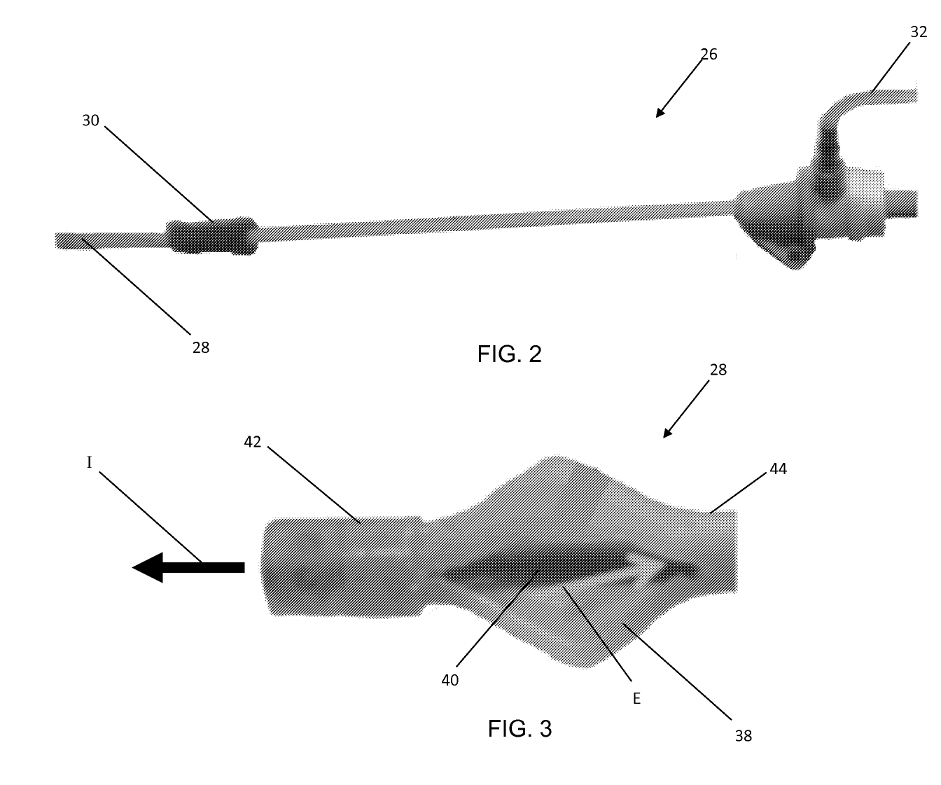

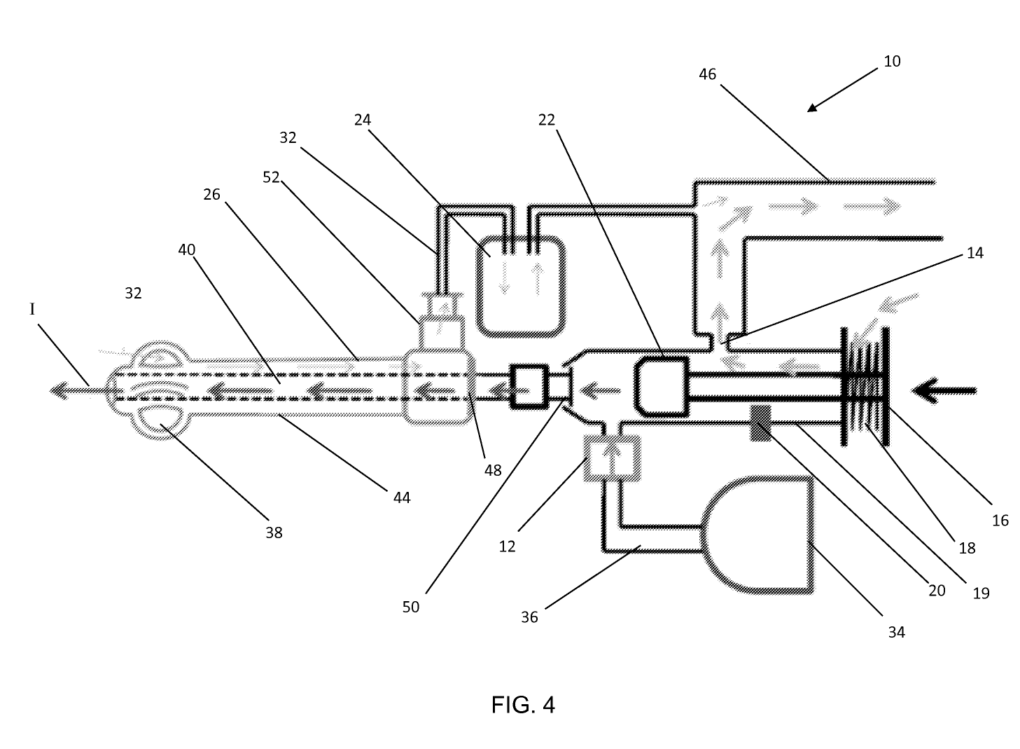

Referring now to the figures, FIG.1 is a picture of an uterotubal irrigation system 10 according to an embodiment of the invention. A syringe 19 with a user controlled plunger 16 is outwardly biased by spring 18, and a plunger seal 22 rests against a plunger stop 20 positioned so that the plunger seal 22 blocks the primary vacuum port 14 on the side of the syringe 19 to the primary vacuum line 46 (see FIG. 4) that is connected to a vacuum source (not shown). A source of injection fluid, such as saline, is stored or held in a fluid reservoir 34, and supplied to the syringe 19 via a supply line 36 and a one way valve 12. A collection tube 24 stores fluids that are evacuated from the uterus via small diameter vacuum line 32 via the irrigation cannula 26. The irrigation cannula 26 is shown in greater detail in FIG. 2 and FIG. 3. At the distal end of the irrigation cannula 26, a cervical plug 30 forms a seal at the cervix os upon insertion of the cannula 26 into the uterus, and allows fluid pressure to be developed in the uterus. The cervical plug may be a balloon, a solid dilated structure on the cannula body, or a foam stopper. FIG. 3 shows an evacuation basket on the distal tip 28 of the irrigation cannula 26. The evacuation basket forms when two or more slits 38 in an external sheath 44 expand outward with the retraction of the irrigation tube 40. The irrigation arrow (I) illustrates the direction of fluid injected via the irrigation tube 40, and evacuation arrow (E) illustrates the direction and entry of fluid withdrawn from the uterus that travels in the space between the external sheath 44 and the irrigation tube 40.

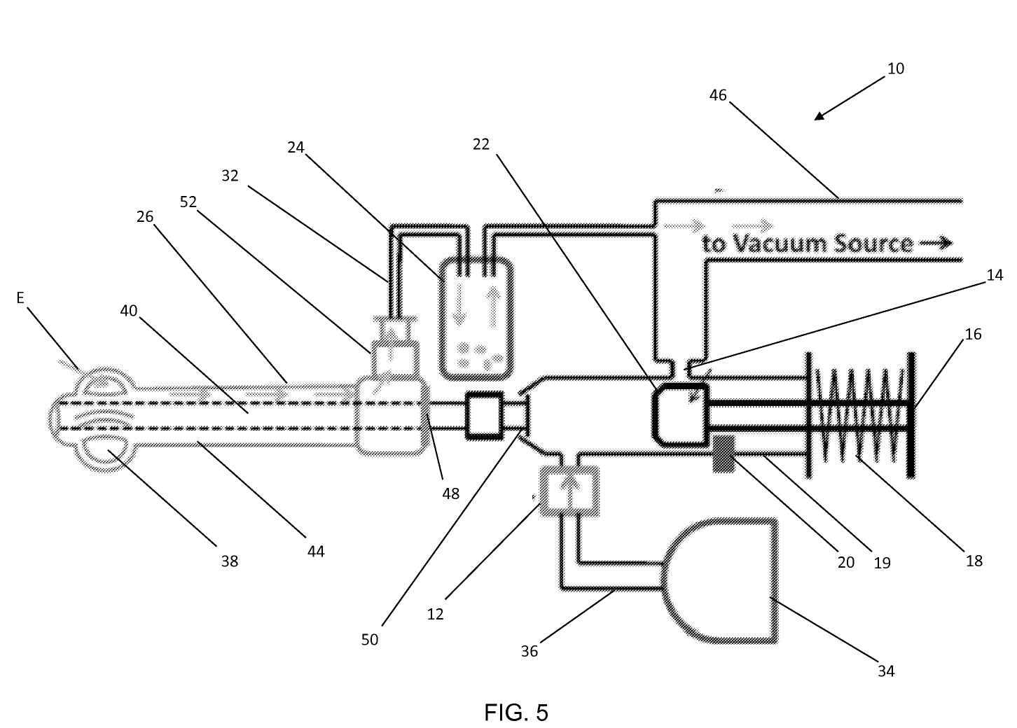

FIG. 4 is a schematic block diagram of the uterotubal irrigation system of FIG. 1 in injection mode according to an embodiment of the invention. With the plunger 16 depressed, the plunger seal 22 is removed from the primary vacuum port 14 on the side of the syringe 19 to the primary vacuum line 46 that is connected to a vacuum source (not shown). In the injection mode, the large diameter vacuum line 46 is open to flow, and there is a resultant minimal vacuum exhibited by the irrigation cannula 26 during fluid injection through the irrigation port 50. FIG. 5 is a schematic block diagram of the uterotubal irrigation system of FIG. 1 in fluid and cell collection mode according to an embodiment of the invention. With the syringe plunger 16 retracted and the large diameter vacuum line 46 is blocked, and vacuum is pulled through the small diameter line 32 to evacuate fluid via the expanded basket formed from the two or more slits 38 on the irrigation cannula 26. The fluid is drawn through the evacuation port 52 and the small diameter vacuum line 32 to the collection tube 24

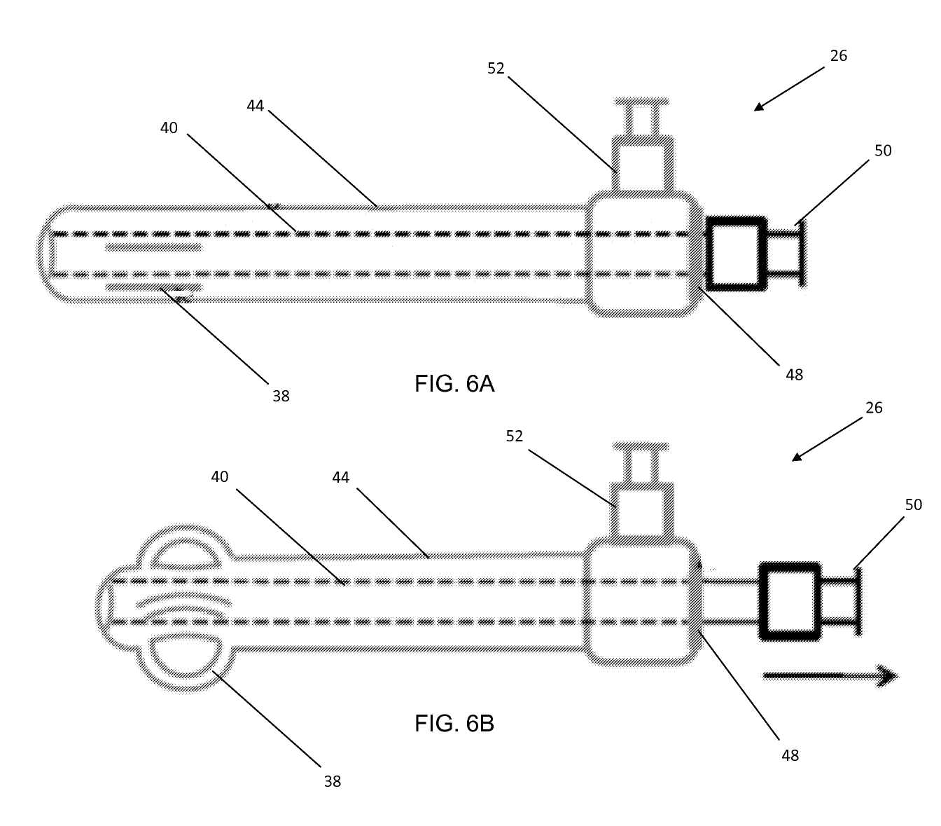

FIGS. 6A and 6B are schematic block diagrams of irrigation cannula 26 of FIG. 1 showing the retraction of the irrigation tube 40 relative to the external sheath 44 to expand the distal evacuation basket formed by the two or more slits 38 according to an embodiment of the invention. The irrigation tube 40 slides on the sliding seal 48 of the evacuation port 52.

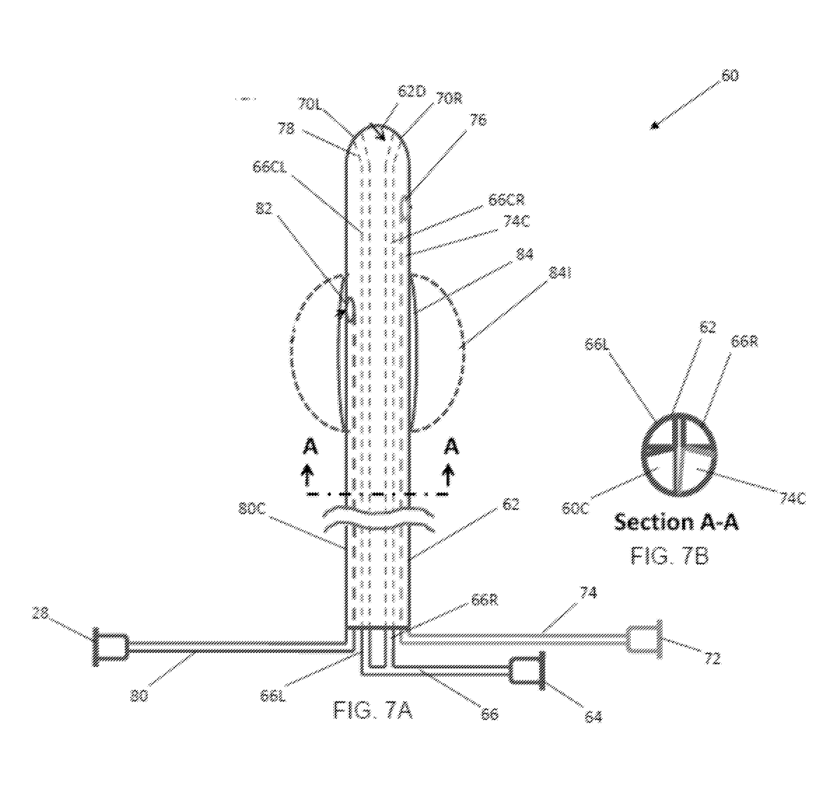

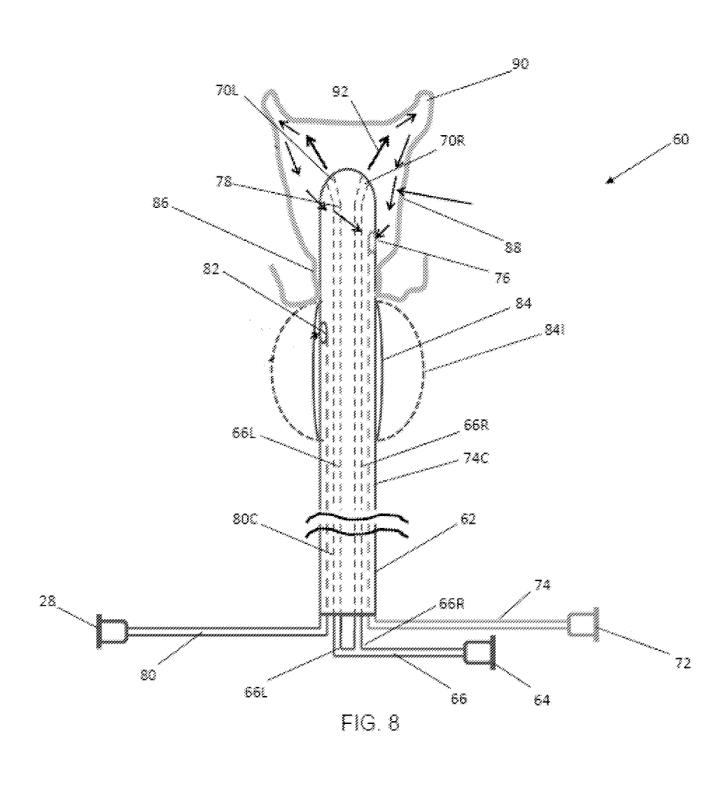

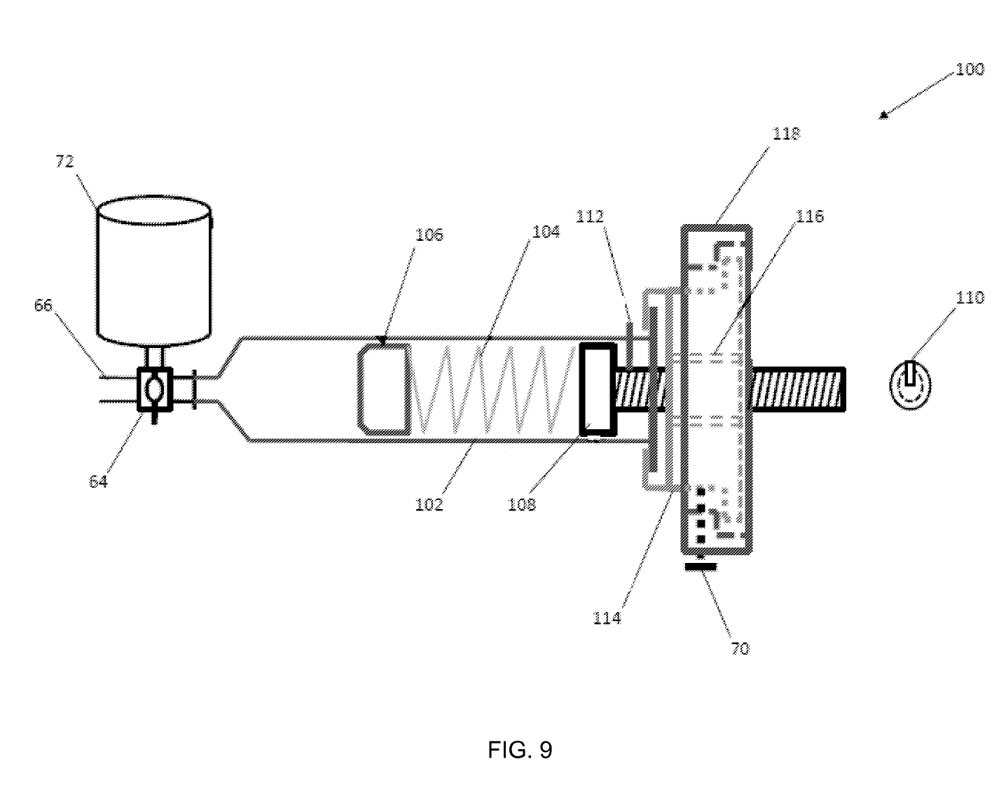

FIG. 7A is a schematic block diagram of a cell collection irrigation catheter 60 according to an embodiment of the invention. The catheter 62 for cell sampling contains two outlet openings (70R, 70L) on the catheter distal tip 62D that inject irrigation fluid in two opposing jets 92 that splay out laterally toward the os or openings 90 of both Fallopian tubes as shown in FIG. 8. The two separate opposing outlets (70R, 70L) on the distal tip 62D of the catheter 62 are angled toward the openings 90 to the Fallopian tubes, where the irrigation channels (66CR, 66CL) within the catheter 62 have a bend 68 that angle the irrigation channels (66CR, 66CL) outward toward the outlet openings (70R, 70L). The irrigation channels (66CR, 66CL) are in fluid communication with irrigation fluid supply lines (66L, 66R), respectively that split off from irrigation fluid supply line 66 that terminates with irrigation port 64. A source of injection fluid, such as saline, is stored or held in a fluid reservoir (see FIG. 9) that connects with the irrigation port 64.

In operation as shown in FIG. 8 an occlusion balloon 84 or a plug that is situated at a distance between 1.5 to 2.5 centimeters proximal to the tip 62D of the catheter 62 is inflated to a full state 841 (as shown by the dotted lines) to seal the cervical os 86 prior to insertion of the catheter tip 62D into the uterine cavity 88. The inflated balloon 841 also serves as a tactile stop to indicate to the physician when to cease the applied inward pressure when inserting the catheter 62 into the uterine cavity 88. The occlusion balloon 84 is inflated with air or gas via supplied inflation outlet opening 82 positioned on the wall of the catheter 62. An inflation channel 80C runs internally along the length of catheter 62 and terminates at the inflation outlet opening 82. The inflation channel 80C is in fluid communication with gas supply line 80 that terminates in balloon inflation port 78. In a specific embodiment, the occlusion balloon 84 or a plug is situated at a distance of 2.0 centimeters proximal to the tip 62D of the catheter 62. The occlusion balloon 84 seals the cervical os 86 during the irrigation and fluid collection process. The injected irrigation fluid, represented by the arrows 92 proceeds a distance into both Fallopian tubes 90, and the fluid then circulates back into the uterine cavity 88, where the fluid exits via a collection inlet 76 in the catheter 62. In an inventive embodiment, the collection port 76 is approximately 1 cm proximal to the distal catheter tip 62D. The collection inlet 76 provides an opening to the collection channel 74C that runs along the inside of the catheter 62. The collection channel 74C is in fluid communication with an external collection line 74 that terminates in a collection port 72. The retrieved irrigation fluid collected at the collection port 72 undergoes cytologic examination to detect the presence of malignant cells. FIG. 7B is a cross-sectional view along line A-A of the cell collection irrigation catheter 62 of FIG. 7A that shows the irrigation channels (66CR, 66CL), the collection channel 74C, and inflation channel 80C within the catheter 62.

FIG. 9 is schematic block diagram of an inventive embodiment of a pressure limiting fluid injection device 100 for use with the cell collection irrigation catheter system 60 of FIGS. 7A, 7B, and 8. It is noted that the pressure limiting fluid injection device 100 may also be used with uterotubal irrigation system 10 that was described in FIGS. 1-6. The inventive fluid injection device 100 is provided that may be used in conjunction with the inventive cell collection irrigation catheter 62 to limit the amount of pressure used for injection. The use of embodiments of the inventive fluid injection device 100 may be used to avoid the pain and discomfort experienced by the patient during a diagnostic procedure.

Embodiments of the pressure limiting fluid injection device 100 of FIG. 9 have a syringe plunger body 102 that is composed of a compression spring 104 connected to the distal sealing plunger face 106. A threaded plunger 108 advances to compress the compression spring 104. The threaded plunger 108 contains a groove 110 along its length that is keyed by a pin 112 that extends through the syringe plunger body 102 into the groove 110. A drive disc 114 is rotatably fixed on the proximal end of the syringe plunger body 102, and the drive disc 114 contains internal threads 116 that mate with the threaded plunger 108. When the drive disc 114 is rotated, the threaded plunger 108 moves forward to compress the fluid inside the syringe plunger body 102. The maximal pressure that may be developed by the syringe is determined by a clutch disc 118 that lies coaxially outside the drive disc 114. At a predetermined amount of torque, the clutch disc 118 slips relative to the drive disc 114. The torque setting may be set by adjusting the friction that exists between the clutch disc 118 and the drive disc 114. In a specific embodiment, one or more clutch adjustment screws 120 extending through the clutch disc 118 may be tightened down on the drive disc 114, so that the torque exerted on the threaded plunger 108 will be limited to a given level. This in turn limits the degree of compression exerted by the compression spring 104, thus limiting the injection pressure. The pressure limiting injection device 100 incorporates the compression spring 114 for energy storage, such that continuous rotation of the clutch disc 118 is unnecessary for fluid injection. Rather, the clutch disc 118 is rotated to bring the pressure limiting fluid injection device 100 to the desired injection pressure level, and then rotated at intervals as necessary to re-pressurize the system. In a specific embodiment the full retraction of the syringe plunger provides a predetermined volume of irrigation fluid (e.g., 5 cc). The output of the pressure limiting injection device 100 is coupled to irrigation port 64 that is in fluid communication with irrigation fluid supply line 66, and a source of injection fluid, such as saline, that is stored or held in a fluid reservoir 72. The irrigation port 64 may act as a check valve which allows fluid to be released from the reservoir 72 on the retraction of the plunger face 56 inside the syringe plunger body 102 which draws in fluid, and closes off the reservoir 72 on a forward stroke of the plunger face 106 that pushes the fluid into the supply line 66 via the irrigation port 64.

The foregoing description is illustrative of particular embodiments of the invention, but is not meant to be a limitation upon the practice thereof. The following claims, including all equivalents thereof, are intended to define the scope of the invention.

1. An uterotubal irrigation system comprising:
a cannula with an external sheath that has a larger inner diameter than an external diameter of an irrigation tube adapted to be positioned within said sheath so as to form an evacuation channel between said external sheath and said irrigation tube along a length of said cannula, and where a distal end of said sheath is connected to a second distal end of said irrigation tube;
a syringe in fluid communication via an irrigation port with said irrigation tube and a fluid reservoir, said syringe having a primary vacuum port connected to a primary vacuum line connected to a vacuum source;
an evacuation port connecting said cannula to said syringe;
a second vacuum line in fluid communication with said evacuation channel and a collection tube, said collection tube for storing a fluid evacuated from a patient's uterus following injection of said fluid that has been previously stored in said fluid reservoir; and
two or more slits formed in a distal end of said sheath, said two or more slits expanding outward with the retraction of said irrigation tube to form an evacuation basket to support uterine walls of the patient's uterus under an applied vacuum during fluid evacuation from the uterus.

2. The system of claim 1 wherein said syringe further comprises a plunger having a plunger seal, where said plunger is biased with a spring so that the plunger seal is positioned to block said primary vacuum port into said syringe, and a vacuum produced by said vacuum source is pulled through said second vacuum line to evacuate said injected fluid via said evacuation basket and said evacuation channel.

3. The system of claim 2 wherein when said plunger is depressed said plunger seal is removed from blocking said primary vacuum port and said primary vacuum line is open to flow, and there is a minimal vacuum supplied by said second vacuum line to said cannula during a resultant fluid injection mode.

4. The system of claim 2 further comprising a stop for the retraction of said plunger at a predetermined volume.

5. The system of claim 2 wherein said plunger modulates a degree of vacuum exhibited in an evacuation mode of said cannula.

6. The system of claim 1 wherein said cannula further comprises an enlarged cervical plug proximal to the distal end of said cannula, said cervical plug forming a seal at a cervix os upon insertion of said cannula into the uterus, and allows a fluid pressure to be developed in the uterus.

7. The system of claim 6 wherein said cervical plug is one of a balloon, a solid dilated structure, or a foam stopper.

8. The system of claim 1 wherein said fluid is saline.

9. The system of claim 1 wherein said fluid reservoir is connected to said syringe via a one way valve.

10. The system of claim 1 wherein said primary vacuum line has a diameter of between 5 to 10 mm, and said second vacuum line has a diameter of 0.5 mm.

11. The system of claim 1 wherein said inner diameter of said external sheath is approximately 0.5 mm greater than the outer diameter of said irrigation tube.

12. The system of claim 1 wherein said irrigation tube slides on a sliding seal of said evacuation port.

13. A process of using the system of claim 1, said process comprising:
inserting said cannula into the patient's uterus;
expanding said evacuation basket by retracing said irrigation tube;
injecting a fluid into the patients uterus;
evacuating said fluid from the patient's uterus and retrieving and collecting said fluid in said collection tube; and
wherein said injecting and evacuating are controlled with the depression of said syringe plunger to modulate the degree of vacuum.

14. The process of claim 13 wherein said injecting and evacuating are staggered to serially and repetitively inject and retrieve multiple fluid aliquots to provide a sufficient fluid volume and number of sample cells for evaluation.

15. The process of claim 13 is a hysterosalpingogram (HSG) procedure.

16. An uterotubal irrigation system comprising:
a catheter with two opposing outlet openings on a distal tip of said catheter that injects an irrigation fluid in two opposing jets that splay out laterally toward the openings of a patient's Fallopian tubes when said catheter is inserted in the uterus of the patient, where said two opposing outlets are angled toward the openings to the patient's Fallopian tubes,
an occlusion balloon or a plug that is situated on a wall of said catheter at a distance between 1.5 to 2.5 centimeters proximal to said distal tip of said catheter that is inflated to seal the patient's cervical os prior to insertion of said catheter distal tip into the patient's uterus; and
a collection inlet proximal to said distal catheter tip for collecting the injected irrigation fluid.

17. The system of claim 16 wherein said catheter further comprises a pair of irrigation channels within said catheter, where each of said irrigation channels have an outward bend that angles said irrigation channels outward toward said outlet openings, where said pair of irrigation channels are in fluid communication with a fluid supply line that terminates in an irrigation port.

18. The system of claim 17 further comprising a pressure limiting fluid injection device in fluid communication with said irrigation port, where said fluid injection device further comprises:
a syringe plunger body containing a compression spring connected to a distal sealing plunger face, where a threaded plunger advances to compress said compression spring and said threaded plunger has a groove along the length of said threaded plunger that is keyed by a pin that extends through said syringe plunger body into said groove;
a drive disc that is rotatably fixed on a proximal end of said syringe plunger body, and where said drive disc has a set of internal threads that mate with said threaded plunger; and
a clutch disc that lies coaxially outside said drive disc; and wherein when said drive disc is rotated, said threaded plunger moves forward to compress the fluid inside said syringe plunger body; and
wherein a maximum pressure level developed by said fluid injection device is determined by said clutch disc; and
wherein at a predetermined set amount of torque, said clutch disc slips relative to said drive disc which limits the degree of compression exerted by said compression spring, and limits a level of injection pressure of said irrigation fluid.

19. The system of claim 18 further comprising one or more clutch adjustment screws extending through said clutch disc that can be tightened down on said drive disc, so that the torque exerted on said threaded plunger will be limited to the predetermined set amount of torque.

20. The system of claim 18 further comprising a fluid reservoir in fluid communication with said irrigation port, where said irrigation port is configured as a check valve which allows the irrigation fluid to be released from said reservoir during a retraction of said plunger face inside said syringe plunger body which draws in the irrigation fluid, and closes off said reservoir on a forward stroke of said plunger face that pushes the irrigation fluid into said supply line via said irrigation port.

21. The system of claim 16 wherein said catheter further comprises an inflation channel that is in fluid communication with a gas supply line that terminates in a balloon inflation port.

22. The system of claim 16 wherein said occlusion balloon or said plug is situated on said wall of said catheter at a distance of 2.0 centimeters proximal to said distal tip of said catheter.

23. The system of claim 16 wherein said catheter further comprises a collection channel that runs along an inside of said catheter, said collection channel in fluid communication with said collection inlet, and where said collection channel is in fluid communication with an external collection line that terminates in a collection port.

24. The system of claim 16 wherein said collection inlet is positioned at a distance of 1.0 centimeter proximal to said distal tip of said catheter.

25. The system of claim 16 wherein said irrigation fluid is saline.

26. A process of using the system of claim 16, said process comprising:
inflating said occlusion balloon;
inserting said catheter into a uterus;
injecting a fluid into the uterus;
evacuating said fluid from said uterus and retrieving; and
collecting said fluid at said collection port.

27. The process of claim 26 wherein said injecting and evacuating are staggered to serially and repetitively inject and retrieve multiple fluid aliquots to provide a sufficient fluid volume and number of sample cells for evaluation.

28. The process of claim 26, wherein said process is a hysterosalpingogram (HSG) procedure.
